(19) United States
(12) Patent Application Publication
(10) Pub. No.: US 2011/0212145 A1
(43) Pub. Date: Sep. 1, 2011

(54) HAIR FIXATIVES COMPRISING CROSS LINKED STARCHES

(75) Inventors: Mark Richard Wheeler, Rockaway, NJ (US); Philip Orawski, Jersey City, NJ (US); Malena Higuera, Edgewater, NJ (US); Lee Mores, Hackettstown, NJ (US)

(73) Assignee: HONEYWELL INTERNATIONAL INC., Morristown, NJ (US)

(21) Appl. No.: 13/036,104

(22) Filed: Feb. 28, 2011

Related U.S. Application Data
(60) Provisional application No. 61/309,145, filed on Mar. 1, 2010.
(60) Provisional application No. 61/391,858, filed on Oct. 11, 2010.

(51) Int. Cl. A61K 8/02; A61K 8/72; A61K 8/73; A61Q 5/06; B82Y 5/00

(52) U.S. Cl. 424/401; 424/70.11; 424/70.13; 977/773; 977/904

(57) ABSTRACT

Hair fixatives such as styling mousse and gel may include biopolymers derived from natural renewable resources. The biopolymer may be cross linked to form a biopolymer latex that may be incorporated into the hair fixatives.

### CROSS-REFERENCE TO RELATED APPLICATIONS

This application claims the benefit of U.S. Provisional Patent Application Ser. No. 61/309,145, filed on Mar. 1, 2010, currently pending, and U.S. Provisional Patent Application Ser. No. 61/391,858, filed on Oct. 11, 2010, currently pending, the disclosures of which are hereby incorporated by reference.

### FIELD OF THE INVENTION

The compositions and processes disclosed herein relate to hair fixatives comprising biopolymers derived from natural resources.

### DESCRIPTION OF RELATED ART

Hair fixatives include styling products such as gels or mousses, which tend to contain polymers that provide structure, allowing a hair style to be molded into shape and to retain that shape once the product is dry. The majority of known fixative polymers are synthetic. However, there is a trend in the personal care industry towards the use of “natural” products, which contain few if any synthetic materials. As used in the personal care industry, the threshold for a “natural” product is that 95% or more by weight of the product is of natural origin.

### SUMMARY OF THE INVENTION

The compositions and processes disclosed herein relate to hair fixatives that include biopolymers derived from natural resources.

In one aspect, hair fixatives are provided that include a biopolymer latex formed from a cross linked biopolymer composition and a carrier composition. The biopolymer being derived from at least one starch obtained from a renewable resource.

In another aspect, a method of producing hair fixatives is provided that includes dispersing a biopolymer into a solvent such as water or a dilute sodium carbonate solution to form a biopolymer latex; and incorporating the biopolymer latex into a carrier composition to form the hair fixative. Prior to contacting the biopolymer and the water, the method may also involve initially wetting the biopolymer with a wetting agent.

### DETAILED DESCRIPTION

Hair fixatives of the present technology include cross linked biopolymer compositions. By “biopolymer” we mean biopolymers such as starch and carbohydrates or other polysaccharides including cellulose, hemicellulose and gums, as well as proteins such as gelatin or whey protein that can be formed into nanoparticles. The biopolymers may be modified using for example cationic groups or carboxymethyl groups by acylation, phosphorylation, hydroxyalkylation, oxidation and the like. Starch and mixtures of starch with other (bio)polymers containing at least 50% starch are preferred. Especially preferred is high-amylopectin starch such as low-amylose starch, i.e. starch having a content of at least 75%, especially at least 90% of amylopectin, such as waxy starch. The biopolymer preferably has a dry substance content of at least 50%, especially at least 60% by weight at the time when processing starts.

Preferably, hair fixatives of the present technology include cross linked biopolymer compositions derived from at least one starch, such as those described in, for example, U.S. Pat. No. 6,677,386 and U.S. Pub. No. 20100143738, both of which are hereby incorporated by reference in their entirety. The at least one starch may be obtained from a variety of natural, renewable resources, including but not limited to, potatoes, corn, wheat, rice, tapioca, or mixtures thereof. In some examples, the starch may be high in amylopectin, and may include, for example at least about 75% amylopectin by weight of the starch, or greater than about 75% amylopectin by weight of the starch, preferably 95% amylopectin. Additionally, chemically modified starches may be used, including for example cationically modified starch such as Emsland cationic potato starch from Kalamazoo Paper Chemicals.

U.S. Pat. No. 6,677,386 describes biopolymer starch nanoparticles which are characterized by an average particle size of less than 400 nanometers. The nanoparticles can be used as a matrix material wherein the matrix material may be a film-forming material, a thickener, a rheology modifier, an adhesive or an adhesive additive (tackifier). U.S. Pub. No 20100143738 describes biolatex conjugate compositions including a biopolymer-additive complex (prepared by co-extruding a biopolymer feedstock, at least one performance-enhancing additive, and at least one plasticizer under shear forces) reacted with a crosslinking agent under shear forces.

The starch may be cross linked in any suitable way to form a cross linked biopolymer. For example, the starch may be cross linked. In such a process, the starch may be cross linked in an extruder, and the extrudate from the extruder may be the cross linked biopolymer. Processes for producing cross linked biopolymers through simultaneous mechanical treatment and cross linking are described generally, for example, in U.S. Pat. Nos. 6,677,386 to Giezen et al, 6,755,915 to Van Soest et al, and 6,921,430 to Bloembergen et al., each of which is hereby incorporated by reference in its entirety. Similar processes may be used in forming cross linked biopolymers. For example, cross linked biopolymers may be produced by dispersing a starch in water, and introducing the starch dispersed in water into a first zone of an extruder.

At least one cross linking agent may be introduced into a second zone of the extruder and combined with the starch dispersed in water. Suitable cross linking agents include, for example, dialdehydes and polyaldehydes, which reversibly form hemiacetals, acid anhydrides and mixed anhydrides (e.g. succinic and acetic anhydride), mixtures thereof and the like. Suitable dialdehydes and polyaldehydes include glutaraldehyde, glyoxal, periodate-oxidized carbohydrates, and the like. Glyoxal is a preferred crosslinker. In some examples, crosslinkers such as epichlorohydrin and other epoxides, triphosphates such as trisodium trimetaphosphate, divinyl sulphone, dialdehydes, thiol reagents, phosphoryl chloride, or an anhydride of a dibasic or polybasic carboxylic acid may be used.

At least one plasticizer may also be present during the cross linking reaction in the extruder. Suitable plasticizers include, but are not limited to, water, polyols (e.g. ethylene glycol, propylene glycol, polyglycols, glycerol, sucrose, maltose, maltodextrines, and sugar alcohols such as sorbitol), urea, sodium lactate, amino acids, citric acid esters, and mixtures thereof. Plasticizers may be used in amounts of from about 5% to about 40% by weight based on the dry weight of the at least one starch. The total amount of plasticizers (i.e. water and additional plasticizer) preferably range from about 5% to about 50% by weight based on the dry weight of the starch or mixture of starch and other biopolymer.

When a cross linking reaction is carried out in an extruder and used to form a cross linked biopolymer as an extrudate, the cross linked biopolymer may be crushed or ground, such as being cryogenically ground, outside the extruder to form biopolymer particles. In some examples, the biopolymer particles may have an average particle size that is from about 0.1 mm to about 10 mm, including, but not limited to from about 0.1 mm to about 5 mm, or from about 0.1 mm to about 3 mm.

The biopolymer particles may then be dispersed in a solvent to form a biopolymer latex. Solvents can include, for example, water or a dilute sodium carbonate solution, including for example solutions containing sodium carbonate in an amount of from about 0.09% to about 0.15% by weight of the solution. Prior to contacting the biopolymer particles and the solvent, the biopolymer particles may be initially wet with a wetting agent. When water is the solvent, the biopolymer latex consists of water swollen biopolymer particles with a size in the range of from about 40 nm to about 100 nm. A biopolymer latex may be prepared with biopolymer contents of up to about 50% by weight of the biopolymer latex, preferably about 30% biopolymer content.

In one example of forming a biopolymer latex, while not required, water may be heated to a temperature of about 40° C. to about 50° C., and from about 0.01% to about 0.09% by weight of sodium carbonate may be dissolved into the heated water to form a dilute sodium carbonate solution. The dilute sodium carbonate solution may be placed under a homogenizer and the biopolymer particles may then be slowly added under continuous homogenization. The homogenizer may be, for example, a rotor stator type homogenizer such as a Silverson mixer. In another example, a Cowles blade affixed to a drill press may also function as a homogenizer. The mixture may be homogenized for sufficient time to ensure complete dispersion of the biopolymer, including but not limited to a time period of up to about 15 minutes or more. Other types of mixing may also be used such as mechanical stirring. A biopolymer latex having a biopolymer content in the range of about 1% to about 50% by weight of the latex may be produced in this manner.

Alternatively, a biopolymer latex may be formed by combining the biopolymer particles with a solvent, such as water, and allowing the combined mixture to stand for a sufficient period of time for the biopolymer to dissolve into the water. Such reactions can be carried out at any suitable temperature, such as from about 20°C to about 70° C., including but not limited to from about 30° C. to about 50° C., and about 43° C. to about 47° C. In some instances, a time period of from about 12 hours to about 72 hours may be sufficient for the biopolymer to disperse into the water without any additional salts or mechanical action.

In examples where the biopolymer particles are initially wet with a wetting agent prior to contacting the solvent, the wetting agents may facilitate the admixture with the solvent. Wetting biopolymer particles with a wetting agent can include by adding the biopolymer particles to a wetting agent, such as, for example, glycerin, propylene glycol, 1,3-propanediol, polyethylene glycols, other suitable polyhydroxy compounds, and combinations thereof, to initially wet the biopolymer. This additional step may shorten the time it takes for the particles to completely disperse in the solvent.

The biopolymer latex may be incorporated into any suitable carrier in order to form a hair fixative of the present technology. Suitable carriers include, but are not limited to gel compositions, foams, emulsions and sprayable liquids. Sprayable liquids may be sprayable, for example, by a pump or aerosol. One example of a suitable gel composition carrier is an aqueous gel. Such gels may be produced by adding a suitable gelling agent to the biopolymer latex. Suitable gelling agents include, but are not limited to, xanthan gum, carrageenan gum, crosslinked polyacrylic acid, cellulosic derivatives, and combinations thereof.

The hair fixatives may further comprise additional components such as plasticizers and/or humectants such as glycerin, propylene glycol, butylene glycol, 1,3-propanediol and similar polyols; preservatives such as DMDM hydantoin, phenoxyethanol, parahydroxybenzoate esters, sodium benzoate, sodium sorbate or others known in the art; conditioning agents such as quaternary ammonium compounds, dimethicone copolyols, cationic polymers, and combinations thereof.

The biopolymer latex may be present in a hair fixative in an amount from about 0.1% by weight of the hair fixative to about 50% by weight of the hair fixative, preferably from about 0.5% by weight of the hair fixative to about 15% by weight of the hair fixative, and more preferably from about 5% by weight of the hair fixative to about 10% by weight of the hair fixative. In one example, a hair fixative comprises about 0.5% to about 50% by weight of a biopolymer latex in an aqueous gel carrier, wherein the biopolymer latex contains from about 30% by weight of the biopolymer.

A biopolymer latex in accordance with the present technology may form films when cast from solution onto polar substrates such as paper or skin. Those skilled in the art will appreciate that these materials may be used in skin care and color cosmetic formulations. For example, such film forming ability may be useful in skin care products such as sunscreen where films aid the spreading and retention of sunscreen actives. Additionally, the ability to form films may be useful in moisturizers where films can allow the skin to retain moisture and so decrease moisture loss, which may improve the Trans Epidermal Water Loss (TEWL) of moisturizers. Another example of an application area where the film formation property of a biopolymer latex of the present technology may be used is in color cosmetics, such as nail polish. A film formed from a biopolymer latex as described herein could be used to form a carrier for adhesion to the nail and dispersion of pigments.

### Example 1

Test Sample A, a hair gel of the present technology containing an aqueous gel carrier and a biopolymer in an amount of about 6% by weight of the hair fixative, was tested in a salon setting against Control Sample B, a hair fixative containing an aqueous gel carrier and a synthetic polymer in an amount of about 6% by weight of the hair fixative.

Product Treatment Protocol: The stylist applied a commercial clarifying shampoo, lathered and rinsed according to the manufacturer's directions. Following the shampoo treatment, the stylist applied a commercial daily conditioner to panelist's wet hair, distributed evenly to the ends and left on the hair for 2 minutes prior to rinsing. After rinsing the conditioner and towel drying hair, the stylist parted the panelist's hair and applied Test Sample A to one side and Control Sample B to the other side. Application of Test Sample A and Control Sample B was randomized between left and right sides of panelist's heads. Equal amounts of Test Sample A and Control Sample B were applied on each side. The gel treated hair was styled according to hair type. Straight-hair panelists were curling iron styled or roller set after blow drying, and curly/wavy panelists were scrunch styled while blow drying.

Panelist Demographics/Style Type: There were a total of 12 panelists. All panelists had color treated hair. 5 panelists had straight hair which was curling iron set except for one panelist which was roller set, 4 panelists had wavy hair (scrunch styled) and 3 panelists had curly hair (scrunch styled).

Evaluation: After applying the test product to the panelists, the stylists rated the products on a 1-5 scale with respect to several subjective factors, with 1 being the worst rating and 5 being the best rating. The panelists also rated the test products in for various subjective factors on a 1-5 scale, providing an initial assessment and an assessment after a time period of about 6-8 hours. The results of the evaluations are provided below.

Stylist Assessment—Test Sample A Vs. Control Sample B:

The mean stylist ratings for Test Sample A and Control Sample B for each product attribute are listed below in Table 1. The differences in the mean ratings were analyzed with respect to statistical significance based upon a 95% confidence interval (p<0.05), which resulted in the following conclusions:
Control Sample B was rated statistically higher in stiffness/crust/hold than Test Sample A.
Control Sample B was rated statistically lower in softness than Test Sample A.
Control Sample B and Test Sample A were a statistical parity in all other attributes; Lack of Tackiness, Curl Definition/Texture, Curl Resiliency/Bounce, Ease of Combing, Lack of Flaking, Lack Frizz, Shine and Body/Volume.

TABLE 1
STYLIST ASSESSMENT

| | Test | Control |
|---|---|---|
| | Sample A | Sample B |
| ATTRIBUTE | (Mean) | (Mean) |
| Lack Tackiness | 4.853 | 4.500 |
| Stiffness/Crust/Hold | 4.167 | 4.833 |
| Curl Definition/Texture | 4.636 | 4.364 |
| Curl Resiliency/Bounce | 4.417 | 4.750 |
| Ease of Combing | 4.333 | 4.167 |
| Lack of Flaking | 4.750 | 4.833 |
| Ease Style Creation/ | 4.583 | 4.417 |
| Manageability | | |
| Soft Feel | 4.500 | 3.917 |
| Lack Frizz | 4.250 | 4.333 |
| Shine | 4.167 | 4.333 |
| Body/Volume | 4.300 | 4.500 |

### Panelist Assessment—Salon Evaluation:

The mean panelist ratings for the initial panelist evaluation of Test Sample A and Control Sample B for each product attribute are listed below in Table 2. The differences in the mean ratings were analyzed with respect to statistical significance based upon a 95% confidence interval (p<0.05), which resulted in the following conclusions:

Control Sample B was rated statistically higher in stiffness/hold than Test Sample A.

Control Sample B was rated statistically lower in soft feel than Test Sample A.

Control Sample B and Test Sample A were a statistical parity in Body/Volume.

TABLE 2
PANELIST ASSESSMENT-INITIAL EVALUATION

| | Test | Control |
|---|---|---|
| | Sample A | Sample B |
| ATTRIBUTE | (Mean) | (Mean) |
| Hold/Stiffness | 3.917 | 4.667 |
| Body/Volume | 4.083 | 4.333 |
| Soft Feel | 4.583 | 3.667 |

### Panelist Assessment—After 6-8 Hours:

The mean panelist ratings for the final panelist evaluation of Test Sample A and Control Sample B for each product attribute are listed below in Table 3. The differences in the mean ratings were analyzed with respect to statistical significance based upon a 95% confidence interval (p<0.05), which resulted in the following conclusions:

Control Sample B was rated statistically higher in stiffness/hold than Test Sample A.

Control Sample B was rated statistically lower in soft feel than Test Sample A.

Control Sample B and Test Sample A were rated parity in Body/Volume.

TABLE 3
PANELIST ASSESSMENT-AFTER 6-8 HOURS

| | Test | Control |
|---|---|---|
| | Sample A | Sample B |
| ATTRIBUTE | (Mean) | (Mean) |
| Hold/Stiffness | 4.000 | 4.750 |
| Body/Volume | 4.333 | 4.083 |
| Soft Feel | 4.583 | 3.667 |

### Example 2

Test Sample A, a hair gel of the present technology containing an aqueous gel carrier and a biopolymer latex in an amount of about 6% by weight of the hair fixative, was bench tested on tresses of hair against Control Sample B, a hair fixative containing an aqueous gel carrier and synthetic polymer in an amount of about 6% by weight of the hair fixative.

Product Treatment Protocol: Tresses were washed with a commercial clarifying shampoo according to manufacturer's directions. Tresses were rinsed and towel-blotted dry for subsequent product application. Five tress replicates were then treated with Test Sample A and five tress replicates were then treated with Control Sample B according to the following steps:
1. Treat each tress with 1 cc gel product and wrap on ‘orange’ convex-shape perm rod (approx. 2 cm diameter, 6.3 cm circumference).
2. Dry wrapped tresses under hood drier for 2 hours until completely dry.
3. Remove curled tress from rod carefully (do not break film) and mount with binder clips to rod.

Evaluation: The evaluation process was conducted according to the following steps:
1. Rod with mounted tresses was placed in an equilibrated chamber set at 90-95% Relative Humidity and 78-82° F. with positioning of top of tress at/near 0″ mark.
2. Measurements of curl length were taken initially, 30 minute intervals up to 4 hrs, 5 hrs, 6 hrs, 24 hrs and 48 hrs. Photographs were taken at each time point for comparison only (camera was aligned at a different vantage point from actual measurements).
3. Curl Retention was calculated using the following formula:

% Curl Retention=(Lₜ−L_{f}/(Lₜ−Lᵢ)×100%
Where:
Lₜ=Length of tress fully extended—5.8″
L_{f}=Length of tress at each time interval
Lᵢ=Length of tress at initial

The curl length measurements and curl retention are provided in Tables 4 and 5 below. As can be seen from the results, Test Sample A had better performance than Control Sample B at time points two hours and beyond.

TABLE 4
Curl Length Measurements

| Time | Al | A2 | A3 | A4 | A5 | Bl | B2 | B3 | B4 | B5 | % RH | T-° F. |
|---|---|---|---|---|---|---|---|---|---|---|---|---|
| 0 | 1.1 | 1.1 | 1.1 | 1.1 | 1 | 1.2 | 1.2 | 1 | 1.1 | 1 | 92 | 79.8 |
| 0.5 | 1.1 | 1.1 | 1.1 | 1.2 | 1 | 1.2 | 1.2 | 1 | 1.1 | 1.1 | 92 | 81 |
| 1 | 1.1 | 1.1 | 1.1 | 1.2 | 1 | 1.2 | 1.2 | 1 | 1.1 | 1.1 | 93 | 81.4 |
| 1.5 | 1.1 | 1.1 | 1.1 | 1.2 | 1 | 1.2 | 1.25 | 1 | 1.25 | 1.2 | 93 | 80.2 |
| 2 | 1.1 | 1.1 | 1.1 | 1.2 | 1 | 1.4 | 1.35 | 1.2 | 1.4 | 1.25 | 93 | 79.4 |
| 2.5 | 1.1 | 1.1 | 1.1 | 1.2 | 1 | 1.5 | 1.4 | 1.25 | 1.5 | 1.35 | 94 | 78.8 |
| 3 | 1.1 | 1.1 | 1.1 | 1.2 | 1 | 1.6 | 1.5 | 1.35 | 1.5 | 1.4 | 94 | 78.2 |
| 3.5 | 1.1 | 1.1 | 1.1 | 1.2 | 1 | 1.75 | 1.6 | 1.4 | 1.65 | 1.4 | 94 | 78.8 |
| 4 | 1.1 | 1.1 | 1.1 | 1.2 | 1 | 2 | 1.65 | 1.5 | 1.75 | 1.5 | 93 | 80.2 |
| 5 | 1.1 | 1.1 | 1.1 | 1.2 | 1 | 2.1 | 1.75 | 1.5 | 1.85 | 1.5 | 93 | 80 |
| 6 | 1.1 | 1.1 | 1.1 | 1.2 | 1 | 2.1 | 1.75 | 1.5 | 1.85 | 1.65 | 93 | 79 |
| 24 | 1.1 | 1.1 | 1.1 | 1.2 | 1 | 2.5 | 2 | 2 | 2.65 | 2.15 | 88 | 75 |
| 48 | 1.1 | 1.1 | 1.1 | 1.2 | 1 | 2.5 | 2 | 2 | 2.65 | 2.25 | 88 | 64 |

TABLE 5
Curl Retention Measurements

| Time | Al | A2 | A3 | A4 | A5 | Mean | Bl | B2 | B3 | B4 | B5 | Mean |
|---|---|---|---|---|---|---|---|---|---|---|---|---|
| 0 | 100.0 | 100.0 | 100.0 | 97.9 | 100.0 | 99.6 | 100.0 | 100.0 | 100.0 | 100.0 | 97.9 | 99.6 |
| 0.5 | 100.0 | 100.0 | 100.0 | 97.9 | 100.0 | 99.6 | 100.0 | 100.0 | 100.0 | 100.0 | 97.9 | 99.6 |
| 1 | 100.0 | 100.0 | 100.0 | 97.9 | 100.0 | 99.6 | 100.0 | 98.9 | 100.0 | 96.8 | 95.8 | 98.3 |
| 1.5 | 100.0 | 100.0 | 100.0 | 97.9 | 100.0 | 99.6 | 100.0 | 98.9 | 100.0 | 96.8 | 95.8 | 98.3 |
| 2 | 100.0 | 100.0 | 100.0 | 97.9 | 100.0 | 99.6 | 95.7 | 96.7 | 95.8 | 93.6 | 94.8 | 95.3 |
| 2.5 | 100.0 | 100.0 | 100.0 | 97.9 | 100.0 | 99.6 | 93.5 | 95.7 | 94.8 | 91.5 | 92.7 | 93.6 |
| 3 | 100.0 | 100.0 | 100.0 | 97.9 | 100.0 | 99.6 | 91.3 | 93.5 | 92.7 | 91.5 | 91.7 | 92.1 |
| 3.5 | 100.0 | 100.0 | 100.0 | 97.9 | 100.0 | 99.6 | 88.0 | 91.3 | 91.7 | 88.3 | 91.7 | 90.2 |
| 4 | 100.0 | 100.0 | 100.0 | 97.9 | 100.0 | 99.6 | 82.6 | 90.2 | 89.6 | 86.2 | 89.6 | 87.6 |
| 5 | 100.0 | 100.0 | 100.0 | 97.9 | 100.0 | 99.6 | 80.4 | 88.0 | 89.6 | 84.0 | 89.6 | 86.3 |
| 6 | 100.0 | 100.0 | 100.0 | 97.9 | 100.0 | 99.6 | 80.4 | 88.0 | 89.6 | 84.0 | 86.5 | 85.7 |
| 24 | 100.0 | 100.0 | 100.0 | 97.9 | 100.0 | 99.6 | 71.7 | 82.6 | 79.2 | 67.0 | 76.0 | 75.3 |
| 48 | 100.0 | 100.0 | 100.0 | 97.9 | 100.0 | 99.6 | 71.7 | 82.6 | 79.2 | 67.0 | 74.0 | 74.9 |

### Example 3

### Formulations

### Formulation 1—6% Biopolymer Latex Hair Gel

| | | Test Sample A | | Control Sample B | |
|---|---|---|---|---|---|
| Phase | Ingredient | % | g(600) | % | g(600) |
| A | Carbopol 980 | 0.6 | 3.6 | 0.6 | 3.6 |
| A | Water | 57.50 | 345 | 57.50 | 345 |
| A | DMDM Hydantoin | 0.3 | 1.8 | 0.3 | 1.8 |
| B | Asensa ® NFF 11 | 20 | 120 | — | — |
| | (30% aqueous) | | | | |
| B | PVP (30% aqueous)* | — | — | 20 | 120 |
| B | Water | 20 | 120 | 20 | 120 |
| C | TEA | 0.8 | 4.8 | 0.6 | 4.8 |
| C | Water | 0.8 | 4.8 | 0.8 | 4.8 |

| | | | | |
|---|---|---|---|---|
| Total | 100 | 600 | 100 | 600 |

*BASF Luviskol K30 from BASF which indicates the degree of polymerization and the molecular weight.

Test Sample A and Control Sample B as tested in Examples 1 and 2 above were prepared by a three phase process. In Phase A, the Carbopol 980 was added to the water and stirred until fully dispersed. The DMDM Hydantoin was then added. As an alternative, the Carbopol may be dispersed under a high shear mixer at low RPM such as about 1000 RPM. In Phase B, the Asensa NFF 11 dispersion and the water were added under gentle agitation, and stirred until fully mixed. In Phase C, the TEA and water were added under continuous agitation, and the mixture was stirred until the gel was fully formed.

Formulation 2—Biolatex Hair Gel with Xanthan Gum

| | | % by |
|---|---|---|
| Phase | Component | weight |
| A | Keltrol CG-SFT (Xanthan Gum) | 1.6 |
| A | Glycerin | 5.0 |
| B | Water | 81.1 |
| B | Asensa ® NFF 11 (30% by weight of biopolymer) | 12 |
| B | Lonza Glydant Plus (preservative) | 0.3 |

A slurry of Phase A was prepared in a beaker. All components of Phase B were combined into a separate beaker and were stirred gently. The Phase A components were then added to the Phase B components under constant mixing. The properties of the resulting hair fixative formulation were as follows:

pH=7.09

Viscosity=6500 Cps. Brookfield DV-II+ Pro, Spindle #4, 20 RPM, 20° C.

Formulation 3—Biolatex Hair Gel with Xanthan Gum and Carrageenan Gum

| | | % by |
|---|---|---|
| Phase | Component | weight |
| A | Keltrol Genuvisco ® NF-131 (Carrageenan gum) | 0.3 |
| A | Keltrol CG-SFT (Xanthan gum) | 0.8 |
| A | Glycerin | 5 |
| B | De-ionized water | 83. |
| B | Asensa ® NFF 11 (30% by weight of polymer) | 10 |
| B | Glydant Plus | 0.1 |

A slurry of Phase A was prepared in a beaker. All components of Phase B were combined into a separate beaker and were stirred gently. The Phase A components were then added to the Phase B components under constant mixing. The properties of the resulting hair fixative formulation were as follows:

pH=7.18

Viscosity=5400 Cps. Brookfield DV-II+ Pro, Spindle #2, 5 RPM, 20° C.

Formulation 4—Biolatex Hair Gel without Predispersion

| Phase | Component | % by weight |
|---|---|---|
| A | Water | 50.16 |
| B | Xanthan Gum | 1.45 |
| B | Glycerin | 1.50 |
| B | 1,3-Propanediol | 1.50 |
| C | Water | 40.00 |
| C | Sodium Carbonate | 0.09 |
| C | Asensa ® NFF 11 biopolymer | 5.00 |
| D | DMDM Hydantoin | 0.30 |

A slurry of Phase B was prepared in a beaker and added to Phase A with stirring. Phase C was mixed in a separate beaker and then added to Phase A/B with stirring. Lastly, Phase D was added to the mixture. The properties of the resulting hair fixative formulation were as follows:

pH=8.4

Viscosity=38,700 cps (Brookfield DV-II+ Pro, Spindle #3, 2.5 RPM, 20° C.

From the foregoing, it will be appreciated that although specific examples have been described herein for purposes of illustration, various modifications may be made without deviating from the spirit or scope of this disclosure. It is therefore intended that the foregoing detailed description be regarded as illustrative rather than limiting, and that it be understood that it is the following claims, including all equivalents, that are intended to particularly point out and distinctly claim the claimed subject matter.

What is claimed is:

1. A hair fixative comprising:
a biopolymer latex formed from a cross linked biopolymer derived from a renewable resource; and
a carrier composition.

2. The hair fixative of claim 1, wherein the biopolymer is derived from starch.

3. The hair fixative of claim 2, wherein the starch comprises greater than about 75% amylopectin.

4. The hair fixative of claim 2, wherein the starch comprises a cationically modified starch.

5. The hair fixative of claim 1, wherein the biopolymer latex comprises particles with a size in the range of from about 40 nm to about 100 nm.

6. The hair fixative of claim 1, wherein the biopolymer latex has a biopolymer content of about 1% to about 50% by weight of the biopolymer latex.

7. The hair fixative of claim 1, wherein the biopolymer is present in the hair fixative in an amount from about 0.1% by weight of the hair fixative to about 50% by weight of the hair fixative.

8. The hair fixative of claim 7, wherein the biopolymer is present in the hair fixative in an amount from about 0.5% by weight of the hair fixative to about 15% by weight of the hair fixative.

9. The hair fixative of claim 7, wherein the biopolymer is present in the hair fixative in an amount from about 5% by weight of the hair fixative to about 10% by weight of the hair fixative.

10. The hair fixative of claim 1, wherein the carrier composition is selected from the group consisting of an aqueous gel, a sprayable liquid, a foam, and an emulsion.

11. A method of producing a hair fixative comprising the steps of:
forming a biopolymer latex; and
incorporating the biopolymer latex into a carrier composition to form the hair fixative.

12. The method of claim 11, wherein the step of forming includes:
dissolving sodium carbonate in water to form a dilute sodium carbonate solution; and
mixing the dilute sodium carbonate solution with a biopolymer to form the biopolymer latex.

13. The method of claim 11, wherein the step of forming includes:
combining a biopolymer and a solvent; and
allowing the combined biopolymer and solvent to stand for a sufficient period of time for the biopolymer to disperse into the water to form a biopolymer latex.

14. The method of producing a hair fixative of claim 13, wherein prior to combining the biopolymer and the solvent the method further comprises:
initially wetting the biopolymer with a wetting agent.

15. The hair fixative of claim 1, further comprising a gelling agent.

16. The hair fixative of claim 1, further comprising a preservative.

17. The hair fixative of claim 1, further comprising a conditioning agent.

18. The hair fixative of claim 1, further comprising a plasticizer.
